SEQUENCE LISTING (22 sequences)

SEQ ID NO: 1 - DNA, 18 bp, Artificial Sequence
  Description of Artificial Sequence Synthetic primer
accatcgcttggtttcgg

SEQ ID NO: 2 - DNA, 20 bp, Artificial Sequence
  Description of Artificial Sequence Synthetic probe
aggcaactgtgctatcccca

SEQ ID NO: 3 - DNA, 20 bp, Artificial Sequence
  Description of Artificial Sequence Synthetic primer
ccccagagacttgttgtagg

SEQ ID NO: 4 - DNA, 20 bp, Artificial Sequence
  Description of Artificial Sequence Synthetic probe
aggcaacggtgctatcccca

SEQ ID NO: 5 - DNA, 18 bp, Artificial Sequence
  Description of Artificial Sequence Synthetic primer
ggaggcaactgtgctatc

SEQ ID NO: 6 - DNA, 26 bp, Artificial Sequence
  Description of Artificial Sequence Synthetic probe
cccatcacggtcatggagtacaccga

SEQ ID NO: 7 - DNA, 17 bp, Artificial Sequence
  Description of Artificial Sequence Synthetic primer
ccgaagacgtccggaaa

SEQ ID NO: 8 - DNA, 25 bp, Artificial Sequence
  Description of Artificial Sequence Synthetic probe
aactgtgctatccccatcacggtca

SEQ ID NO: 9 - DNA, 20 bp, Artificial Sequence
  Description of Artificial Sequence Synthetic primer
cccagagacttgttgtagga

SEQ ID NO: 10 - DNA, 19 bp, Artificial Sequence
  Description of Artificial Sequence Synthetic primer
gagatgctgctggccttca

SEQ ID NO: 11 - DNA, 25 bp, Artificial Sequence
  Description of Artificial Sequence Synthetic probe
tgaccttcgtcaagcagtacggccc

SEQ ID NO: 12 - DNA, 22 bp, Artificial Sequence
  Description of Artificial Sequence Synthetic primer
ccttgtagatctccgtcagctt

SEQ ID NO: 13 - DNA, 19 bp, Artificial Sequence
  Description of Artificial Sequence Synthetic primer
ccacctcctcgatcgagtt

SEQ ID NO: 14 - DNA, 32 bp, Artificial Sequence
  Description of Artificial Sequence Synthetic probe
cgctgtatgtggttatacgtaaactgcagtcg

SEQ ID NO: 15 - DNA, 16 bp, Artificial Sequence
  Description of Artificial Sequence Synthetic primer
tgcgccccagcatgtc

SEQ ID NO: 16 - DNA, 18 bp, Artificial Sequence
  Description of Artificial Sequence Synthetic primer
gtccgctccggagaagac

SEQ ID NO: 17 - DNA, 19 bp, Artificial Sequence
  Description of Artificial Sequence Synthetic probe
tccctgtgtcggccaccgc

SEQ ID NO: 18 - DNA, 19 bp, Artificial Sequence
  Description of Artificial Sequence Synthetic primer
gcgcttgggtcgactgagg

SEQ ID NO: 19 - DNA, 16 bp, Artificial Sequence
  Description of Artificial Sequence Synthetic primer
gtcggttccgcgcttg

SEQ ID NO: 20 - DNA, 10 bp, Artificial Sequence
  Description of Artificial Sequence Synthetic probe
aaacacgtgc

SEQ ID NO: 21 - DNA, 10 bp, Artificial Sequence
  Description of Artificial Sequence Synthetic probe
ccttgttcca

SEQ ID NO: 22 - DNA, 10 bp, Artificial Sequence
  Description of Artificial Sequence Synthetic probe
cagggacgat

(19) United States
(12) Patent Application Publication
(10) Pub. No.: US 2013/0302784 A1
(43) Pub. Date: Nov. 14, 2013

(54) OPTIMIZED PROBES AND PRIMERS AND METHODS OF USING SAME FOR THE BINDING, DETECTION, DIFFERENTIATION, ISOLATION AND SEQUENCING OF HERPES SIMPLEX VIRUS

(71) Applicant: INTELLIGENT MEDICAL DEVICES, INC., Cambridge, MA (US)

(72) Inventors: Heinz Reiske, Framingham, MA (US); Chesley Leslin, Boston, MA (US); William Lauzier, East Bridgewater, MA (US); Alice A. Jacobs, Cambridge, MA (US)

(21) Appl. No.: 13/851,626

(22) Filed: Mar. 27, 2013

Related U.S. Application Data
(60) Provisional application No. 61/617,977, filed on Mar. 30, 2012.
(60) Provisional application No. 61/644,349, filed on May 8, 2012.

(51) Int. Cl. C12Q 1/70

(52) U.S. Cl. CPC C12Q 1/705
USPC 435/5; 536/23.1; 536/25.3; 536/24.32; 536/24.33

(57) ABSTRACT

Described herein are primers and probes useful for the binding, detecting, differentiating, isolating, and sequencing of HSV-1 and/or HSV-2 viruses.

### RELATED APPLICATIONS

This application claims the benefit of U.S. Provisional Application No. 61/617,977, filed on Mar. 30, 2012 and U.S. Provisional Application No. 61/644,349, filed on May 8, 2012, the contents of which are incorporated by reference herein in their entirety.

### BACKGROUND

Herpes simplex viruses (HSV) are enveloped, double stranded DNA viruses of the family Herpesviridae. Herpes simplex viruses are divided into two distinct types: herpes simplex virus-1 (HSV-1) and herpes simplex virus-2 (HSV-2). HSV-1 mostly causes cold sores and fever blisters, generally around the mouth, and keratitis in the eyes. HSV-2 usually causes genital lesions and spreads through sexual contact and skin-to-skin contact.

HSV-1 and HSV-2 cycle between productive and latent stages of infection. At the productive stage, infection of epithelial cells presents clinically as lesions, especially on mucosal surfaces, that can last for several weeks. After initial infection, both types of HSVs can enter sensory nerve endings and maintain themselves in the nuclei of dorsal root ganglia cells, establishing latency. Upon reactivation, HSV migrates along the ganglia cell's axon and infects epithelial cells.

HSV contributes to or causes several diseases, including herpes keratitis, orofacial herpes and genital herpes. One of the most serious complications of HSV infection, usually from HSV-1, is Herpes Simplex Encephalitis (HSE), a severe viral infection of the human central nervous system. HSE usually occurs upon reactivation of a latent HSV infection and generally affects individuals under the age of 20 and over age 40. Newborn encephalitis can occur by HSV-2 transmission from the infected mother to the neonate. Studies have suggested that HSV-1 may also contribute to Alzheimer's disease.

Traditional testing for herpes simplex virus is performed using viral culture methods. Currently, the majority of herpes simplex virus testing is performed using cell culture, serological assays, or direct fluorescent antibody testing.

Herpes simplex virus detection would allow for improved treatments of viral infections. A rapid and accurate diagnostic test panel for the simultaneous detection and differentiation (typing) of HSV-1 and HSV-2 virus, therefore, would provide clinicians with an effective tool for identifying patients symptomatic for one or more of the HSV viruses and subsequently supporting effective treatment regimens.

### SUMMARY

The present disclosure provides compositions and assays for detecting the presence of herpes simplex virus (HSV-1 and/or HSV-2).

Described herein are nucleic acid probes and primers for binding, detecting, differentiating, isolating and sequencing all or the majority of known, characterized variants of HSV-1 and/or HSV-2, with a high degree of sensitivity and specificity. The above described assay also includes an internal control.

A diagnostic test or tests that detect and distinguish between HSV-1 and HSV-2 simultaneously in humans is important because such detection is critical in early patient identification and treatment. The assays described herein also aid in the intervention of the spread of these highly infectious viruses.

Many facilities utilize viral culture-based methods for the determination and detection of respiratory infections, which requires days to obtain the results. The methods of detection of the present invention described herein can be carried out within a minimal number of hours, allowing clinicians to rapidly determine the appropriate treatment options for individuals infected with herpes simplex virus(es).

One embodiment is directed to an isolated nucleic acid sequence comprising a sequence selected from the group consisting of: SEQ ID NOS: 1-19.

One embodiment is directed to a method of hybridizing one or more isolated nucleic acid sequences comprising a sequence selected from the group consisting of: SEQ ID NOS: 1-19 to an HSV-1 and/or HSV-2 sequence, comprising contacting one or more isolated nucleic acid sequences to a sample comprising the HSV-1 and/or HSV-2 sequence under conditions suitable for hybridization. In a particular embodiment, the sequence is a genomic sequence, a naturally occurring plasmid, a naturally occurring transposable element, a template sequence or a sequence derived from an artificial construct. In a particular embodiment, the method(s) further comprise isolating and/or sequencing the hybridized HSV-1 and/or HSV-2 sequence.

One embodiment is directed to a primer set for amplifying DNA of HSV-1 and/or HSV-2 comprising at least one forward primer selected from the group consisting of SEQ ID NOS: 1, 5, 7, 10, 13 and 16; and at least one reverse primer selected from the group consisting of SEQ ID NOS: 3, 9, 12, 15, 18 and 19.

One embodiment is directed to a primer set (at least one forward primer and at least one reverse primer) selected from the group consisting of: Groups 1-8 of Table 3.

One embodiment is directed to a method of producing a nucleic acid product, comprising contacting one or more isolated nucleic acid sequences selected from the group consisting of SEQ ID NOS: 2, 4, 6, 8, 11, 14 and 17 to a sample comprising an HSV-1 and/or HSV-2 sequence under conditions suitable for nucleic acid polymerization. In a particular embodiment, the nucleic acid product is an HSV-1 and/or HSV-2 amplicon produced using at least one forward primer selected from the group consisting of SEQ ID NOS: 1, 5, 7, 10, 13 and 16 and at least one reverse primer selected from the group consisting of SEQ ID NOS: 3, 9, 12, 15, 18 and 19.

One embodiment is directed to a probe that hybridizes to an amplicon produced as described herein, e.g., using the primers described herein. In a particular embodiment, the probe comprises a sequence selected from the group consisting of SEQ ID NOS: 2, 4, 6, 8, 11, 14 and 17. In a particular embodiment, the probe(s) is labeled with a detectable label selected from the group consisting of: a fluorescent label, a chemiluminescent label, a quencher, a radioactive label, biotin and gold.

One embodiment is directed to a set of probes that hybridize to an amplicon produced as described herein, e.g., using the primers described herein. In a particular embodiment, a first probe can comprise an HSV-1 sequence, for example, selected from the group consisting of SEQ ID NOS: 2, 4, 6 and 8, and a second probe can comprise an HSV-2 sequence, for example, selected from the group consisting of SEQ ID NOS: 11, 14 and 17.

One embodiment is directed to a set of probes that hybridize to an amplicon produced as described herein, e.g., using the primers described herein. In a particular embodiment, a first probe can comprise an HSV-1 sequence, for example, selected from the group consisting of SEQ ID NOS: 2, 4, 6 and 8; a second probe can comprise an HSV-2 sequence, for example, selected from the group consisting of SEQ ID NOS: 11, 14 and 17 and a third probe can comprise an internal control sequence. In a particular embodiment, each of the probes is labeled with a different detectable label. In additional embodiments, one or more of the probes is labeled with the same detectable label.

One embodiment is directed to a probe that hybridizes directly to the genomic sequences of the target without amplification. In a particular embodiment, the probe comprises a sequence, for example, selected from the group consisting of SEQ ID NOS: 2, 4, 6, 8, 11, 14 and 17. In a particular embodiment, the probe(s) is labeled with a detectable label, for example, selected from the group consisting of: a fluorescent label, a chemiluminescent label, a quencher, a radioactive label, biotin and gold.

One embodiment, using any of the probe combinations described herein, is directed to a set of probes that hybridize directly to the genomic sequences of the target without amplification.

In one embodiment, the probe(s) is fluorescently labeled and the step of detecting the binding of the probe to the amplified product comprises measuring the fluorescence of the sample. In one embodiment, the probe comprises a fluorescent reporter moiety and a quencher of fluorescence-quenching moiety. Upon probe hybridization with the amplified product, the exonuclease activity of a DNA polymerase dissociates the probe's fluorescent reporter and the quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. In another embodiment, each probe in the multiplex reaction is labeled with a different distinguishable and detectable label.

In a particular embodiment, the probes are molecular beacons. Molecular beacons are single-stranded probes that form a stem-and-loop structure. A fluorophore is covalently linked to one end of the stem and a quencher is covalently linked to the other end of the stem forming a stem hybrid; fluorescence is quenched when the formation of the stem loop positions the fluorophore proximal to the quencher. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein may be designed and utilized as molecular beacons.

One embodiment is directed to a method for detecting HSV-1 and/or HSV-2 DNA in a sample, comprising: (a) contacting the sample with at least one forward primer comprising a sequence selected from the group consisting of: SEQ ID NOS: 1, 5 and 7 (HSV-1); 10, 13 and 16 (HSV-2); and at least one reverse primer comprising a sequence selected from the group consisting of: SEQ ID NO: 3 and 9 (HSV-1); 12, 15, 18 and 19 (HSV-2); under conditions such that nucleic acid amplification occurs to yield an amplicon; and (b) contacting the amplicon with one or more probes comprising one or more sequences selected from the group consisting of: SEQ ID NOS: 2, 4, 6 and 8 (HSV-1); 11, 14 and 17 (HSV-2), under conditions such that hybridization of the probe to the amplicon occurs, wherein hybridization of the probe is indicative of HSV-1 and/or HSV-2 DNA in the sample.

In a particular embodiment, each of the one or more probes is labeled with a different detectable label. In a particular embodiment, the one or more probes are labeled with the same detectable label. In a particular embodiment, the sample is selected from the group consisting of: saliva, fluids collected from the ear, eye, mouth, and respiratory airways, sputum, tears, oropharyngeal swabs, nasopharyngeal swabs, throat swabs, nasopharyngeal aspirates, bronchoalveolar lavage fluid, skin swabs, lip swabs, genital swabs, rectal swabs, cerebrospinal fluid, anogenital or oral lesion swabs, bone marrow, nasal aspirates, nasal wash, and fluids and cells obtained by the perfusion of tissues of both human and animal origin. In one embodiment, the sample is from a human, is non-human in origin, or is derived from an inanimate object or environmental surfaces. In a particular embodiment, the at least one forward primer, the at least one reverse primer and the one or more probes are selected from the group consisting of: Groups 1-8 of Table 3. In a particular embodiment, the method(s) further comprise isolating and/or sequencing the HSV-1 and/or HSV-2 DNA.

One embodiment is directed to a primer set or collection of primer sets for amplifying DNA of an HSV-1 strain, comprising a nucleotide sequence selected from the group consisting of: (1) SEQ ID NOS: 1 and 3; and (2) SEQ ID NOS: 5 and 3; and (3) SEQ ID NOS: 7 and 9.

One embodiment is directed to a primer set or collection of primer sets for amplifying DNA of an HSV-2 strain, comprising a nucleotide sequence selected from the group consisting of: (1) SEQ ID NOS: 10 and 12; (2) SEQ ID NOS: 13 and 15; (3) SEQ ID NOS: 16 and 18 and (4) SEQ ID NOS: 16 and 19.

One embodiment is directed to the simultaneous detection and differentiation in a multiplex format of HSV-1 and HSV-2.

One embodiment is directed to a primer set or collection of primer sets for amplifying DNA of HSV-1 and HSV-2 simultaneously, comprising:

(a) (1) SEQ ID NOS: 1 and 3; (2) SEQ ID NOS: 5 and 3 and (3) SEQ ID NOS: 7 and 9 (forward and reverse primers for amplifying DNA of HSV-1); and

(b) (1) SEQ ID NOS: 10 and 12; (2) SEQ ID NOS: 13 and 15; (3) SEQ ID NOS: 16 and 18 and (4) SEQ ID NOS: 16 and 19 (forward and reverse primers for amplifying DNA of HSV-2).

A particular embodiment is directed to oligonucleotide probes for binding to DNA of HSV-1 and/or HSV-2, comprising a nucleotide sequence selected from the group consisting of SEQ ID NOS: 2, 4, 6, 8 (HSV-1 probes) and 11, 14 and 17 (HSV-2 probes).

One embodiment is directed to a kit for detecting DNA of an HSV-1 and/or HSV-2 virus in a sample, comprising one or more probes comprising a sequence selected from the group consisting of: SEQ ID NOS: 2, 4, 6, 8 (HSV-1 probes) 11, 14 and 17 (HSV-2 probes). In a particular embodiment, the kit further comprises internal control probes. In a particular embodiment, the kit further comprises a) at least one forward internal control primer; and b) at least one internal control reverse primer. In a particular embodiment, the kit further comprises reagents for isolating and/or sequencing the DNA in the sample. In a particular embodiment, the one or more probes are labeled with different detectable labels. In a particular embodiment, the one or more probes are labeled with the same detectable labels. In a particular embodiment, the at least one forward primer, the at least one reverse primer and the one or more probes are selected from the group consisting of: Groups 1-8 of Table 3.

One embodiment is directed to a method for diagnosing a condition, symptom or disease in a human associated with an HSV-1 and/or HSV-2 virus, comprising: a) contacting a sample with at least one forward and reverse primer set selected from the group consisting of: Groups 1-8 of Table 3; b) conducting an amplification reaction, thereby producing an amplicon; and c) detecting the amplicon using one or more probes selected from the group consisting of: SEQ ID NOS: 2, 4, 6, 8 (HSV-1) 11, 14 and 17 (HSV-2); wherein the generation of an amplicon is indicative of the presence of an HSV-1 and/or HSV-2 virus in the sample. In a particular embodiment, the sample is saliva, fluids collected from the ear, eye, mouth, and respiratory airways, sputum, tears, oropharyngeal swabs, nasopharyngeal swabs, throat swabs, nasopharyngeal aspirates, bronchoalveolar lavage fluid, skin swabs, lip swabs, genital swabs, rectal swabs, cerebrospinal fluid, anogenital or oral lesion swabs, bone marrow, nasal aspirates, nasal wash, and fluids and cells obtained by the perfusion of tissues of both human and animal origin. In one embodiment, the sample is from a human, is non-human in origin, or is derived from an inanimate object or environmental surfaces. A sample may be collected from more than one collection site, e.g., genital and rectal swabs. In a particular embodiment, the complications, conditions, symptoms or diseases in humans associated with an HSV-1 and/or HSV-2 virus are selected from the group consisting of: fever, sore throat, sore mouth, gingivial lesions, lip lesions, ulcerative lesions, vesicular lesions, gingivostomatitis, edema, localized lymphadenopathy, anorexia, malaise, pharyngitis, dysuria, macules, papules, genital ulcers, encephalitis, lethargy, seizures, keratoconjunctivitis, meningitis, myelitis and radiculitis.

One embodiment is directed to a kit for amplifying and sequencing DNA of an HSV-1 and/or HSV-2 virus in a sample, comprising: a) at least one forward primer or primer pair comprising the sequence selected from the group consisting of: SEQ ID NOS: 1, 5 and 7 (HSV-1); 10, 13 and 16 (HSV-2); and b) at least one reverse primer or primer pair comprising the sequence selected from the group consisting of: SEQ ID NOS: 3 and 9 (HSV-1); 12, 15, 18 and 19 (HSV-2); and c) reagents for the sequencing of amplified DNA fragments.

The oligonucleotides of the present invention and their resulting amplicons do not cross react and, thus, will work together without negatively impacting each other. The primers and probes to detect HSV-1 and/or HSV-2 do not cross react with each other. The primers and probes of the present invention do not cross react with other potentially contaminating species that would be present in a sample matrix.

### DETAILED DESCRIPTION

A diagnostic test or tests that can simultaneously detect and differentiate HSV-1 and HSV-2 is important, as herpes simplex virus infections are common world-wide and potentially lead to very serious outcomes, including herpes simplex encephalitis.

Described herein are optimized probes and primers that, alone or in various combinations, allow for the amplification, detection, differentiation, isolation, and sequencing of HSV-1 and/or HSV-2 viruses that can be found in clinical isolates. Specific probes and primers, i.e., probes and primers that can detect all or a majority of known and characterized strains of HSV-1 and/or HSV-2, have been discovered and are described herein. Nucleic acid primers and probes for detecting specific HSV-1 and/or HSV-2 genetic material and methods for designing and optimizing the respective primer and probe sequences are described herein.

The primers and probes of the present invention can be used for the detection of HSV-1 and/or HSV-2, without loss of assay precision or sensitivity. The primers and probes described herein can be used, for example, to identify and/or confirm symptomatic patients for the presence of HSV-1 and/or HSV-2 viruses in a multiplex format

### Herpes Simplex Virus

HSV has a double-stranded DNA genome packaged in an icosadeltahedral capsid. A layer of proteins, designated as the tegument, surrounds the capsid. The outer envelope of the virus is a lipid bilayer which contains at least 10 viral glycoproteins. These glycoproteins mediate the attachment and subsequent entry of HSV into eukaryotic cells. Whitley, RJ and Roizman, B, Herpes Simplex Viruses, In: Clinical Virology, 2^{nd} ed, Richman, D D; Whitley, R J (Eds), ASM Press, Washington, D.C., 2002, p. 375.

Glycoprotein D binds to cellular receptors, such as herpesvirus entry mediator (HVEM) and is necessary for entry into the cell. Studies suggest that Glycoprotein D may also modulate the host immune response. Stiles, K M; Whitbeck, J C; Lou, H; Cohen, G H; Eisenberg, R J; Krummenacher, C, Herpes Simplex Virus Glycoprotein D Interferes with Binding of Herpesvirus Entry Mediator to Its Ligand through Downregulation and Direct Competition, J. Virol., 84(22): 11646-11660 (2010). Glycoprotein D from HSV-1 differs from HSV-2 in that it can bind to several different receptors, including the 3-0 Heparan Sulfate receptor. (Akhtar, J and Shukla, D, Viral entry mechanisms: cellular and viral mediators of herpes simplex virus entry, FEBS J., 276(24): 7228-7236 (2009).

Glycoprotein B plays an important role in viral attachment and entry into cells. Glycoprotein B interacts with cell surface heparan sulfate proteoglycan and recent studies suggest interaction with other cell surface receptors. Bender, F C; Whitbeck, J C; Lou, H; Cohen, G H; Eisenberg, R J, Herpes Simplex Virus Glycoprotein B Binds to Cell Surfaces Independently of Heparan Sulfate and Blocks Virus Entry. J. Virol. 79(18): 11588-97 (2005).

HSV DNA Polymerase is composed of a catalytic subunit (UL30) and a processivity factor (UL42). The DNA polymerase is required for viral replication.

Herpes simplex virus is quite common among the adult population. More than 90 percent of adults have antibodies directed against HSV-1 by middle age and reports indicate 22 percent of the U.S. population has antibodies directed against HSV-2. HSV infections can occur throughout the year and reactivation can be quite frequent. Moreover, reactivation can occur subclinically without obvious symptoms. Corey, L, Herpes Simplex Viruses In: Harrison's Principles of Internal Medicine, 14^{th} ed., Fauci, A S; Braunwald, E; Isselbacher, K J; Wilson, J D; Martin, J B; Kasper, D L; et al. (Eds.), McGraw Hill, New York, 1998, p. 1080-1086.

A variety of symptoms are associated with HSV infection. Primary HSV-1 infection commonly manifests itself as gingivostomatitis and pharyngitis. Secondary or recurrent HSV-1 infection presents itself usually as herpes labialis, infection of the lip. Ulcerative lesions can also develop on the posterior pharynx and/or tonsillar pillars. Genital infections characterized by lesions on the external genitalia also occur. Symptoms include fever, headache, malaise, myalgias, pain, itching, dysuria and vaginal and urethral discharge. Recurrence of HSV-2 genital infection occurs more frequently than HSV-1 genital infections. HSV can also infect the finger (Herpes Whitlow) causing abrupt edema, erythema and localized tenderness of the infected area. In addition, HSV can infect other areas of the skin (herpes gladiatorum). HSV is the most frequent cause of corneal blindness. Symptoms include acute onset of pain, blurring of vision, chemosis, conjunctivitis and dendritic lesions of the cornea. The most serious HSV disease is viral encephalitis, infection of the central nervous system. HSV-1 usually causes this disease and is estimated to account for 10 to 20 percent of all encephalitis cases. HSV-2 commonly causes neonatal infections, resulting from contact by the neonate with infected genital secretions.

### Assays

Table 1 demonstrates various possible diagnostic outcome scenarios using the probes and primers described herein in diagnostic methods.

TABLE 1

| Target | Expected Results | | | | |
|---|---|---|---|---|---|
| HSV-1 | + | + | − | − | − |
| HSV-2 | + | − | + | − | − |
| IC | +/− | +/− | +/− | + | − |
| Interpretation | HSV-1/HSV-2 | HSV-1 | HSV-2 | None | Invalid |

+, target detected;
−, target not detected;
HSV-1 corresponding to the herpes simplex virus-1 strain;
HSV-2 corresponding to the herpes simplex virus-2 strain;
IC corresponding to the internal control.

Detection of the internal control (IC) indicates that the sample result is valid, where an absence of a signal corresponding to the IC indicates either an invalid result or that one or more of the specific targets is at a high starting concentration. A signal indicating a high starting concentration of specific target in the absence of an IC signal is considered to be a valid sample result.

The advantages of a multiplex format are: (1) simplified and improved testing and analysis; (2) increased efficiency and cost-effectiveness; (3) decreased turnaround time (increased speed of reporting results); (4) increased productivity (less equipment time needed); and (5) coordination/standardization of results for patients for multiple organisms (reduces error from inter-assay variation).

Detection of HSV-1 and/or HSV-2 can lead to earlier and more effective treatment of a subject. The methods for diagnosing and detecting HSV-1 and/or HSV-2 viruses described herein can be coupled with effective treatment therapies (e.g., antivirals). The treatments for such infections will depend upon the clinical disease state of the patient, as determinable by one of skill in the art.

The present invention therefore provides a method for specifically detecting in a sample the presence of two herpes simplex viruses using the primers and probes provided herein. Of particular interest in this regard is the ability of the disclosed primers and probes, as well as those that can be designed according to the disclosed methods, to specifically detect all or a majority of presently characterized strains of known, characterized HSV variants. The optimized primers and probes are useful, therefore, for identifying and diagnosing HSV-1 and/or HSV-2 infection, whereupon an appropriate treatment can then be administered to the individual to eradicate the virus(es).

The present invention provides one or more sets of primers that can anneal to all currently identified HSV-1 and/or HSV-2 strains and thereby amplify a target from a biological sample. The present invention provides, for example, at least a first primer and at least a second primer for HSV-1 and/or HSV-2, each of which comprises a nucleotide sequence designed according to the inventive principles disclosed herein, which are used together to amplify DNA from HSV-1 and/or HSV-2 in a mixed-flora sample in a multiplex assay.

Also provided herein are probes that hybridize to the HSV-1 and/or HSV-2 sequences and/or amplified products derived from the HSV-1 and/or HSV-2 sequences. A probe can be labeled, for example, such that when it binds to an amplified or unamplified target sequence, or after it has been cleaved after binding, a fluorescent signal is emitted that is detectable under various spectroscopy and light measuring apparatuses. The use of a labeled probe, therefore, can enhance the sensitivity of detection of a target in an amplification reaction of DNA of HSV-1 and/or HSV-2 because it permits the detection of viral-derived DNA at low template concentrations that might not be conducive to visual detection as a gel-stained amplification product.

Primers and probes are sequences that anneal to a viral genomic or viral genomic derived sequence, e.g., the HSV strains (the “target” sequence). The target sequence can be, for example, an anti-viral resistance mutation or a viral genome. In one embodiment, the entire gene sequence can be “scanned” for optimized primers and probes useful for detecting the anti-viral resistance mutation or the viral genome. In other embodiments, particular regions of the genome can be scanned, e.g., regions that are documented in the literature as being useful for detecting multiple genes, regions that are conserved, or regions where sufficient information is available in, for example, a public database, with respect to the antibiotic resistance genes.

Sets or groups of primers and probes are generated based on the target to be detected. The set of all possible primers and probes can include, for example, sequences that include the variability at every site based on the known viral genome, or the primers and probes can be generated based on a consensus sequence of the target. The primers and probes are generated such that the primers and probes are able to anneal to a particular sequence under high stringency conditions. For example, one of skill in the art recognizes that for any particular sequence, it is possible to provide more than one oligonucleotide sequence that will anneal to the particular target sequence, even under high stringency conditions. The set of primers and probes to be sampled includes, for example, all such oligonucleotides for all known and characterized HSV viruses. Alternatively, the primers and probes include all such oligonucleotides for a given consensus sequence for a target.

Typically, stringent hybridization and washing conditions are used for nucleic acid molecules over about 500 bp. Stringent hybridization conditions include a solution comprising about 1 M Na⁺ at 25° C. to 30° C. below the Tm; e.g., 5×SSPE, 0.5% SDS, at 65° C.; see, Ausubel, et al., Current Protocols in Molecular Biology, Greene Publishing, 1995; Sambrook et al., Molecular Cloning: A Laboratory Manual, Cold Spring Harbor Press, 1989). Tm is dependent on both the G+C content and the concentration of salt ions, e.g., Na⁺ and K⁺. A formula to calculate the Tm of nucleic acid molecules greater than about 500 bp is Tm=81.5+0.41(% (G+C))−log₁₀[Na⁺]. Washing conditions are generally performed at least at equivalent stringency conditions as the hybridization. If the background levels are high, washing can be performed at higher stringency, such as around 15° C. below the Tm.

The set of primers and probes, once determined as described above, are optimized for hybridizing to a plurality of viral genes by employing scoring and/or ranking steps that provide a positive or negative preference or “weight” to certain nucleotides in a target nucleic acid strain sequence. If a consensus sequence is used to generate the full set of primers and probes, for example, then a particular primer sequence is scored for its ability to anneal to the corresponding sequence of every known native target sequence. Even if a probe were originally generated based on a consensus, the validation of the probe is in its ability to specifically anneal and detect every, or a large majority of, target sequences. The particular scoring or ranking steps performed depend upon the intended use for the primer and/or probe, the particular target nucleic acid sequence, and the number of variant genes of that target nucleic acid sequence. The methods of the invention provide optimal primer and probe sequences because they hybridize to all or a subset of HSV viruses. Once optimized oligonucleotides are identified that can anneal to such genes, the sequences can then further be optimized for use, for example, in conjunction with another optimized sequence as a “primer set” or for use as a probe. A “primer set” is defined as at least one forward primer and one reverse primer.

Described herein are methods for using the primers and probes for producing a nucleic acid product, for example, comprising contacting one or more nucleic acid sequences of SEQ ID NOS: 1-19 to a sample comprising the HSV-1 and/or HSV-2 strain under conditions suitable for nucleic acid polymerization. The primers and probes can additionally be used to sequence the DNA of HSV-1 and/or HSV-2, or used as diagnostics to, for example, detect the HSV-1 and/or HSV-2 in a clinical isolate sample, e.g., obtained from a subject, e.g., a mammalian subject. Particular combinations for amplifying DNA of HSV-1 and/or HSV-2 include, for example, using at least one forward primer selected from the group consisting of: 1, 5, 7, 10, 13 and 16; and at least one reverse primer selected from the group consisting of SEQ ID NOS: 3, 9, 12, 15, 18 and 19.

Methods are described for detecting HSV-1 and/or HSV-2 in a sample, for example, comprising (1) contacting at least one forward and reverse primer set, e.g., SEQ ID NOS: 1, 5, 7, 10, 13 and 16 (forward primers); and 3, 9, 12, 15, 18 and 19 (reverse primers) to a sample; (2) conducting an amplification; and (3) detecting the generation of an amplified product, wherein the generation of an amplified product indicates the presence of HSV-1 and/or HSV-2 pathogens in a clinical isolate sample.

The detection of amplicons using probes described herein can be performed, for example, using a labeled probe, e.g., the probe comprising a nucleotide sequence selected from the group consisting of: SEQ ID NOS: 2, 4, 6, 8, 11, 14 and 17 that hybridizes to one of the strands of the amplicon generated by at least one forward and reverse primer set. The probe(s) can be, for example, fluorescently labeled, thereby indicating that the detection of the probe involves measuring the fluorescence of the sample of the bound probe, e.g., after bound probes have been isolated. Probes can also be fluorescently labeled in such a way, for example, such that they only fluoresce upon hybridizing to their target, thereby eliminating the need to isolate hybridized probes. The probe can also comprise a fluorescent reporter moiety and a quencher of fluorescence moiety. Upon probe hybridization with the amplified product, the exonuclease activity of a DNA polymerase can be used to dissociate the probe's reporter and quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. For multiplex reactions involving more than one distinct probe, each of the probes can be labeled with a different distinguishable and detectable label.

The probes can be molecular beacons. Molecular beacons are single-stranded probes that form a stem-loop structure. A fluorophore can be, for example, covalently linked to one end of the stem and a quencher can be covalently linked to the other end of the stem forming a stem hybrid. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein can be modified and utilized as molecular beacons.

The probes can be conjugated to a minor groove binder (MGB) group. This increases the stability of the probe template hybrid and reduces the tolerance for mismatches, which results in better discriminatory properties. With MGBs, the added functionality is due to a peptide moiety conjugated to the nucleic acid sequence that alters the binding properties of the probe.

The probes can alternatively be modified using locked nucleic acid (LNA) technology (see Kaur, H. et al., Biochemistry, 45:7347-55, 2006; and You, Y. et al., Nucl. Acids Res., 34:e60, 2006). LNA is a modified nucleic acid that is incorporated into the probe, replacing one or more of the nucleotides, thus altering the way that region of the probe binds to its complementary target sequence. A LNA, often referred to as inaccessible RNA, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. The locked ribose conformation enhances base stacking and backbone pre-organization. This significantly increases the thermal stability (melting temperature) of oligonucleotides.

Primer or probe sequences can be ranked according to specific hybridization parameters or metrics that assign a score value indicating their ability to anneal to viral strains under highly stringent conditions. Where a primer set is being scored, a “first” or “forward” primer is scored and the “second” or “reverse”-oriented primer sequences can be optimized similarly but with potentially additional parameters, followed by an optional evaluation for primer dimers, for example, between the forward and reverse primers.

The scoring or ranking steps that are used in the methods of determining the primers and probes include, for example, the following parameters: a target sequence score for the target nucleic acid sequence(s), e.g., the PriMD® score; a mean conservation score for the target nucleic acid sequence(s); a mean coverage score for the target nucleic acid sequence(s); 100% conservation score of a portion (e.g., 5′ end, center, 3′ end) of the target nucleic acid sequence(s); a species score; a strain score; a subtype score; a serotype score; an associated disease score; a year score; a country of origin score; a duplicate score; a patent score; and a minimum qualifying score. Other parameters that are used include, for example, the number of mismatches, the number of critical mismatches (e.g., mismatches that result in the predicted failure of the sequence to anneal to a target sequence), the number of native strain sequences that contain critical mismatches, and predicted Tm values. The term “Tm” refers to the temperature at which a population of double-stranded nucleic acid molecules becomes half-dissociated into single strands. Methods for calculating the Tm of nucleic acids are known in the art (Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory).

The resultant scores represent steps in determining nucleotide or whole target nucleic acid sequence preference, while tailoring the primer and/or probe sequences so that they hybridize to a plurality of target nucleic acid sequences. The methods of determining the primers and probes also can comprise the step of allowing for one or more nucleotide changes when determining identity between the candidate primer and probe sequences and the target nucleic acid sequences, or their complements.

In another embodiment, the methods of determining the primers and probes comprise the steps of comparing the candidate primer and probe nucleic acid sequences to “exclusion nucleic acid sequences” and then rejecting those candidate nucleic acid sequences that share identity with the exclusion nucleic acid sequences. In another embodiment, the methods comprise the steps of comparing the candidate primer and probe nucleic acid sequences to “inclusion nucleic acid sequences” and then rejecting those candidate nucleic acid sequences that do not share identity with the inclusion nucleic acid sequences.

In other embodiments of the methods of determining the primers and probes, optimizing primers and probes comprises using a polymerase chain reaction (PCR) penalty score formula comprising at least one of a weighted sum of: primer Tm—optimal Tm; difference between primer Tms; amplicon length—minimum amplicon length; and distance between the primer and a TagMan® probe. The optimizing step also can comprise determining the ability of the candidate sequence to hybridize with the most target nucleic acid strain sequences (e.g., the most target organisms or genes). In another embodiment, the selecting or optimizing step comprises determining which sequences have mean conservation scores closest to 1, wherein a standard of deviation on the mean conservation scores is also compared.

In other embodiments, the methods further comprise the step of evaluating which target nucleic acid sequences are hybridized by an optimal forward primer and an optimal reverse primer, for example, by determining the number of base pair differences between target nucleic acid sequences in a database. For example, the evaluating step can comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; and/or (4) determining the secondary structure of the forward primer, reverse primer, and/or target. In an embodiment, the evaluating step includes evaluating whether the forward primer sequence, reverse primer sequence, and/or probe sequence hybridizes to sequences in the database other than the nucleic acid sequences that are representative of the target strains.

The present invention provides oligonucleotides that have preferred primer and probe qualities. These qualities are specific to the sequences of the optimized probes, however, one of skill in the art would recognize that other molecules with similar sequences could also be used. The oligonucleotides provided herein comprise a sequence that shares at least about 60-70% identity with a sequence described in Table 3. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequences of Table 3 or complement thereof. The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art.

In addition, the sequences, including those provided in Table 3 and sequences sharing certain sequence identities with those in Table 3, as described above, can be incorporated into longer sequences, provided they function to specifically anneal to and identify viral strains. In one aspect, the longer sequences have 1, 2, 3, 4, 5, 6, 7, 8, 9, or 10 additional bases at either or both ends of the original sequences. These longer sequences are also within the scope of the present disclosure.

The primer and/or probe nucleic acid sequences of the invention are complementary to the target nucleic acid sequence. The probe and/or primer nucleic acid sequences of the invention are optimal for identifying numerous strains of a target nucleic acid, e.g., HSV viruses. In an embodiment, the nucleic acids of the invention are primers for the synthesis (e.g., amplification) of target nucleic acid sequences and/or probes for identification, isolation, detection, or analysis of target nucleic acid sequences, e.g., an amplified target nucleic acid that is amplified using the primers of the invention.

The present oligonucleotides hybridize with more than one HSV type (as determined by differences in its sequence). The probes and primers provided herein can, for example, allow for the detection of currently identified HSV types or a subset thereof. In addition, the primers and probes of the present invention, depending on the HSV sequence(s), can allow for the detection of previously unidentified HSV sequences. The methods of the invention provide for optimal primers and probes, and sets thereof, and combinations of sets thereof, which can hybridize with a larger number of targets than available primers and probes.

In other aspects, the invention also provides vectors (e.g., plasmid, phage, expression), cell lines (e.g., mammalian, insect, yeast, bacterial, viral), and kits comprising any of the sequences of the invention described herein. The invention further provides known or previously unknown target nucleic acid strain sequences that are identified, for example, using the methods of the invention. In an embodiment, the target nucleic acid sequence is an amplification product. In another embodiment, the target nucleic acid sequence is a native or synthetic nucleic acid. The primers, probes, and target nucleic acid sequences, vectors, cell lines, and kits can have any number of uses, such as diagnostic, investigative, confirmatory, monitoring, predictive or prognostic.

Diagnostic kits that comprise one or more of the oligonucleotides described herein, which are useful for screening for and/or detecting the presence of HSV-1 and/or HSV-2 in an individual and/or from a sample, are provided herein. An individual can be a human male, human female, human adult, human child, or human fetus. A sample includes any item, surface, material, clothing, or environment, in which it may be desirable to test for the presence of herpes simplex virus(es). Thus, for instance, the present invention includes testing door handles, faucets, table surfaces, elevator buttons, chairs, toilet seats, sinks, kitchen surfaces, children's cribs, bed linen, pillows, keyboards, and so on, for the presence of herpes simplex virus(es).

A probe of the present invention can comprise a label such as, for example, a fluorescent label, a chemiluminescent label, a radioactive label, biotin, gold, dendrimers, aptamer, enzymes, proteins, quenchers and molecular motors. In an embodiment, the probe is a hydrolysis probe, such as, for example, a TagMan® probe. In other embodiments, the probes of the invention are molecular beacons, any fluorescent probes, probes modified with locked nucleic acids and probes that are replaced by any double stranded DNA binding dyes (e.g., SYBR Green® 1).

Oligonucleotides of the present invention do not only include primers that are useful for conducting the aforementioned amplification reactions, but also include oligonucleotides that are attached to a solid support, such as, for example, a microarray, multiwell plate, column, bead, glass slide, polymeric membrane, glass microfiber, plastic tubes, cellulose, and carbon nanostructures. Hence, detection of herpes simplex viruses can be performed by exposing such an oligonucleotide-covered surface to a sample such that the binding of a complementary strain DNA sequence to a surface-attached oligonucleotide elicits a detectable signal or reaction.

Oligonucleotides of the present invention also include primers for isolating and sequencing nucleic acid sequences derived from any identified or yet to be isolated and identified HSV virus.

One embodiment of the invention uses solid support-based oligonucleotide hybridization methods to detect gene expression. Solid support-based methods suitable for practicing the present invention are widely known and are described (PCT application WO 95/11755; Huber et al., Anal. Biochem., 299:24, 2001; Meiyanto et al., Biotechniques, 31:406, 2001; Relogio et al., Nucleic Acids Res., 30:e51, 2002; the contents of which are incorporated herein by reference in their entirety). Any solid surface to which oligonucleotides can be bound, covalently or non-covalently, can be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one common approach, the solid support is coated with a polymeric layer that provides linker arms with a plurality of reactive ends/sites. A common example of this type is glass slides coated with polylysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption/ionization time of flight (MALDI-TOF) mass spectrometry. Therefore, streptavidin can be covalently attached to a solid support, and a biotinylated nucleic acid molecule will bind to the streptavidin-coated surface. In one version of this method, an amino-coated silicon wafer is reacted with the n-hydroxysuccinimido-ester of biotin and complexed with streptavidin. Biotinylated oligonucleotides are bound to the surface at a concentration of about 20 fmol DNA per mm².

One can alternatively directly bind DNA to the support using carbodiimides, for example. In one such method, the support is coated with hydrazide groups, and then treated with carbodiimide. Carboxy-modified nucleic acid molecules are then coupled to the treated support. Epoxide-based chemistries are also being employed with amine modified oligonucleotides. Other chemistries for coupling nucleic acid molecules to solid substrates are known to those of skill in the art.

The nucleic acid molecules, e.g., the primers and probes of the present invention, must be delivered to the substrate material, which is suspected of containing or is being tested for the presence of herpes simplex virus(es). Because of the miniaturization of the arrays, delivery techniques must be capable of positioning very small amounts of liquids in very small regions, very close to one another and amenable to automation. Several techniques and devices are available to achieve such delivery. Among these are mechanical mechanisms (e.g., arrayers from GeneticMicroSystems, MA, USA) and ink jet technology. Very fine pipets can also be used.

Other formats are also suitable within the context of this invention. For example, a 96-well format with fixation of the nucleic acids to a nitrocellulose or nylon membrane can also be employed.

After the nucleic acid molecules have been bound to the solid support, it is often useful to block reactive sites on the solid support that are not consumed in binding to the nucleic acid molecule. In the absence of the blocking step, excess primers and/or probes can, to some extent, bind directly to the solid support itself, giving rise to non-specific binding. Non-specific binding can sometimes hinder the ability to detect low levels of specific binding. A variety of effective blocking agents (e.g., milk powder, serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays, that can be used to simultaneously observe the expression of a number of herpes simplex virus(es). Oligonucleotide arrays comprise two or more oligonucleotide probes provided on a solid support, wherein each probe occupies a unique location on the support. The location of each probe can be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting gene expression can be made and used according to conventional techniques described (Lockhart et al., Nat. Biotech., 14:1675-1680, 1996; McGall et al., Proc. Natl. Acad. Sci. USA, 93:13555, 1996; Hughes et al., Nat. Biotechnol., 19:342, 2001). A variety of oligonucleotide array designs are suitable for the practice of this invention.

Generally, a detectable molecule, also referred to herein as a label, can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR. PCR primers used in generating the probes are chosen, for example, based on the sequences of Table 3. In one embodiment, oligonucleotide control probes also are used. Exemplary control probes can fall into at least one of three categories referred to herein as (1) normalization controls, (2) expression level controls and (3) negative controls. In microarray methods, one or more of these control probes can be provided on the array with the inventive HSV gene-related oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. The normalization controls also allow for the semi-quantification of the signals from other features on the microarray. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (i.e., without forming secondary structures) and do not match any test probes. In one embodiment, the normalization controls are viral genes.

“Negative control” probes are not complementary to any of the test oligonucleotides (i.e., the HSV oligonucleotides), normalization controls, or expression controls. In one embodiment, the negative control is a mammalian, viral or bacterial gene that is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In one embodiment, background is calculated as the average hybridization signal intensity for the lowest 5 to 10 percent of the oligonucleotide probes being used, or, where a different background signal is calculated for each target gene, for the lowest 5 to 10 percent of the probes for each gene. Where the oligonucleotide probes corresponding to a particular target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. A probe can be labeled with an enzyme or, alternatively, the probe is labeled with a moiety that is capable of binding to another moiety that is linked to the enzyme. For example, in the biotin-streptavidin interaction, the streptavidin is conjugated to an enzyme such as horseradish peroxidase (HRP). A chromogenic substrate is added to the reaction and is processed/cleaved by the enzyme. The product of the cleavage forms a color, either in the UV or visible spectrum. In another embodiment, streptavidin alkaline phosphatase can be used in a labeled streptavidin-biotin immunoenzymatic antigen detection system.

The invention also provides methods of labeling nucleic acid molecules with cleavable mass spectrometry tags (CMST; U.S. Patent Application NO: 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene. See also Kochanowski, Quantitative PCR Protocols (Humana Press, 1999); Innis et al., supra.; Vandesompele et al., Genome Biol., 3: RESEARCH0034, 2002; Stein, Cell Mol. Life Sci. 59:1235, 2002.

The forward and reverse amplification primers and internal hybridization probe is designed to hybridize specifically and uniquely with one nucleotide sequence derived from the transcript of a target gene. In one embodiment, the selection criteria for primer and probe sequences incorporates constraints regarding nucleotide content and size to accommodate TaqMan® requirements. SYBR Green® can be used as a probe-less Q-RTPCR alternative to the TaqMan®-type assay, discussed above (ABI Prism® 7900 Sequence Detection System User Guide Applied Biosystems, chap. 1-8, App. A-F. (2002)). A device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867).

The primers and probes of the present invention may anneal to or hybridize to various HSV genetic material or genetic material derived therefrom, or other genetic material derived therefrom, such as RNA, DNA, cDNA, or a PCR product.

A “sample” that is tested for the presence of HSV-1 and/or HSV-2 includes, but is not limited to a tissue sample, such as, for example, saliva, fluids collected from the ear, eye, mouth, and respiratory airways, sputum, tears, oropharyngeal swabs, nasopharyngeal swabs, throat swabs, nasopharyngeal aspirates, bronchoalveolar lavage fluid, skin swabs, lip swabs, genital swabs, rectal swabs, cerebrospinal fluid, anogenital or oral lesion swabs, bone marrow, nasal aspirates, nasal wash, and fluids and cells obtained by the perfusion of tissues of both human and animal origin. The tissue sample may be fresh, fixed, preserved, or frozen. A sample also includes any item, surface, material, or clothing, or environment, in which it may be desirable to test for the presence of HSV-1 and/or HSV-2. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of HSV-1 and/or HSV-2.

The target nucleic acid strain that is amplified may be RNA or DNA or a modification thereof. Thus, the amplifying step can comprise isothermal or non-isothermal reactions, such as polymerase chain reaction, Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isothermal amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. The detection step can also comprise the incorporation of a label (e.g., fluorescent or radioactive) during an extension reaction. The detecting step comprises measuring fluorescence, mass, charge, and/or chemiluminescence.

The target nucleic acid strain may not need amplification and may be RNA or DNA or a modification thereof. If amplification is not necessary, the target nucleic acid strain can be denatured to enable hybridization of a probe to the target nucleic acid sequence.

Hybridization may be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Scoring a Set of Predicted Annealing Oligonucleotides

Each of the sets of primers and probes selected is ranked by a combination of methods as individual primers and probes and as a primer/probe set. This involves one or more methods of ranking (e.g., joint ranking, hierarchical ranking, and serial ranking) where sets of primers and probes are eliminated or included based on any combination of the following criteria, and a weighted ranking again based on any combination of the following criteria, for example: (A) Percentage Identity to Target Strains; (B) Conservation Score; (C) Coverage Score; (D) Strain/Subtype/Serotype Score; (E) Associated Disease Score; (F) Duplicates Sequences Score; (G) Year and Country of Origin Score; (H) Patent Score, and (I) Epidemiology Score.

### (A) Percentage Identity

A percentage identity score is based upon the number of target nucleic acid strain (e.g., native) sequences that can hybridize with perfect conservation (the sequences are perfectly complimentary) to each primer or probe of a primer set and probe set. If the score is less than 100%, the program ranks additional primer set and probe sets that are not perfectly conserved. This is a hierarchical scale for percent identity starting with perfect complimentarity, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus sequence and these scores represent how many of the target nucleic acid strains sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid strain sequences have the same base (either an A, C, G, or T/U) at that position. If there is an equal number of bases (e.g., 50% A & 50% T) at a position, it is identified with an N.

(ii) Candidate Primer/Probe Sequence Conservation

An overall conservation score is generated for each candidate primer or probe sequence that represents how many of the target nucleic acid strain sequences will hybridize to the primers or probes. A candidate sequence that is perfectly complimentary to all the target nucleic acid strain sequences will have a score of 1.0 and rank the highest. For example, illustrated below in Table 2 are three different 10-base candidate probe sequences that are targeted to different regions of a consensus target nucleic acid strain sequence. Each candidate probe sequence is compared to a total of 10 native sequences.

TABLE 2
(SEQ ID NO: 20)

| | |
|---|---|
| #1 | A A A C A C G T G C |
| | 0.7 1.0 1.0 1.0 1.0 1.0 1.0 1.0 1.0 1.0 |

→Number of target nucleic acid strain sequences that are perfectly
complimentary-7. Three out of the ten sequences do not have an A at
position 1.
(SEQ ID NO: 21)

| | |
|---|---|
| #2 | C C T T G T T C C A |
| | 1.0 0.9 1.0 0.9 0.9 1.0 1.0 1.0 1.0 1.0 |

→Number of target nucleic acid strain sequences that are perfectly
complimentary-7, 8, or 9. At least one target nucleic acid strain does
not have a C at position 2, T at position 4, or G at position 5. These
differences may all be on one target nucleic acid strain molecule or may
be on two or three separate molecules.
(SEQ ID NO: 22)

| | |
|---|---|
| #3 | C A G G G A C G A T |
| | 1.0 1.0 1.0 1.0 1.0 0.9 0.8 1.0 1.0 1.0 |

→Number of target nucleic acid strain sequences that are perfectly
complimentary-7 or 8. At least one target nucleic acid strain does not
have an A at position 6 and at least two target nucleic acid strain do
not have a C at position 7. These differences may all be on one target
nucleic acid strain molecule or may be on two separate molecules.

A simple arithmetic mean for each candidate sequence would generate the same value of 0.97. The number of target nucleic acid strain sequences identified by each candidate probe sequence, however, can be very different. Sequence #1 can only identify 7 native sequences because of the 0.7 (out of 1.0) score by the first base—A. Sequence #2 has three bases each with a score of 0.9; each of these could represent a different or shared target nucleic acid strain sequence. Consequently, Sequence #2 can identify 7, 8 or 9 target nucleic acid strain sequences. Similarly, Sequence #3 can identify 7 or 8 of the target nucleic acid strain sequences. Sequence #2 would, therefore, be the best choice if all the three bases with a score of 0.9 represented the same 9 target nucleic acid strain sequences.

(iii) Overall Conservation Score of the Primer and Probe Set—Percent Identity

The same method described in (ii) when applied to the complete primer set and probe set will generate the percent identity for the set (see A above). For example, using the same sequences illustrated above, if Sequences #1 and #2 are primers and Sequence #3 is a probe, then the percent identity for the target can be calculated from how many of the target nucleic acid sequences are identified with perfect complementarity to all three primer/probe sequences. The percent identity could be no better than 0.7 (7 out of 10 target nucleic acid strain sequences) but as little as 0.1 if each of the degenerate bases reflects a different target nucleic acid strain sequence. Again, an arithmetic mean of these three sequences would be 0.97. As none of the above examples were able to capture all the target nucleic acid strain sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complementary to all the known native sequences that were used to generate the consensus sequence. If there is no such candidate, then the sets are ranked according to how many degenerate bases can be accepted and still hybridize to just the target sequence during the PCR and yet identify all the native sequences.

The hybridization conditions, for TagMan® as an example, are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers and probe set. The rules can be as simple as counting the number of degenerate bases to more sophisticated approaches based on exploiting the PCR criteria used by the PriMD® software. Each target nucleic acid strain sequence has a value or weight (see Score assignment above). If the failed target nucleic acid strain sequence is medically valuable, the primer/probe set is rejected. This in silico analysis provides a degree of confidence for a given genotype and is important when new sequences are added to the databases. New target nucleic acid strain sequences are automatically entered into both the “include” and “exclude” categories. Published primer and probes will also be ranked by the PriMD software.

(iv) Position (5′ to 3′) of the Base Conservation Score

In an embodiment, primers do not have bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria are different. For example, with a TagMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TagMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TagMan® probe. If there are two or more degenerate bases in a primer and probe set the ranking will initially select the sets where the degeneracies occur on different sequences.

### B. Coverage Score

The total number of aligned sequences is considered under a coverage score. A value is assigned to each position based on how many times that position has been reported or sequenced. Alternatively, coverage can be defined as how representative the sequences are of the known strains, subtypes, etc., or their relevance to a certain diseases. For example, the target nucleic acid strain sequences for a particular gene may be very well conserved and show complete coverage but certain strains are not represented in those sequences.

A sequence is included if it aligns with any part of the consensus sequence, which is usually a whole gene or a functional unit, or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

### C. Strain/Subtype/Serotype Score

A value is assigned to each strain or subtype or serotype based upon its relevance to a disease. For example, viral strains and/or species that are linked to high frequencies of infection will have a higher score than strains that are generally regarded as benign. The score is based upon sufficient evidence to automatically associate a particular strain with a disease. For example, certain strains of adenovirus are not associated with diseases of the upper respiratory system. Accordingly, there will be sequences included in the consensus sequence that are not associated with diseases of the upper respiratory system.

### D. Associated Disease Score

The associated disease score pertains to strains that are not known to be associated with a particular disease (to differentiate from D above). Here, a value is assigned only if the submitted sequence is directly linked to the disease and that disease is pertinent to the assay.

### E. Duplicate Sequences Score

If a particular sequence has been sequenced more than once it will have an effect on representation, for example, a strain that is represented by 12 entries in GenBank of which six are identical and the other six are unique. Unless the identical sequences can be assigned to different strains/subtypes (usually by sequencing other gene or by immunology methods) they will be excluded from the scoring.

### F. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. Certain divergent strains from more obscure countries or sources may also be less relevant to the locations that will likely perform clinical tests, or may be more important for certain countries (e.g., North America, Europe, or Asia).

### G. Patent Score

Candidate target strain sequences published in patents are searched electronically and annotated such that patented regions are excluded. Alternatively, candidate sequences are checked against a patented sequence database.

### H. Minimum Qualifying Score

The minimum qualifying score is determined by expanding the number of allowed mismatches in each set of candidate primers and probes until all possible native sequences are represented (e.g., has a qualifying hit).

### I. Other

A score is given to based on other parameters, such as relevance to certain patients (e.g., pediatrics, immunocompromised) or certain therapies (e.g., target those strains that respond to treatment) or epidemiology. The prevalence of an organism/strain and the number of times it has been tested for in the community can add value to the selection of the candidate sequences. If a particular strain is more commonly tested then selection of it would be more likely. Strain identification can be used to select better vaccines.

### Example 2

### Primer/Probe Evaluation

Once the candidate primers and probes have received their scores and have been ranked, they are evaluated using any of a number of methods of the invention, such as BLAST analysis and secondary structure analysis.

### A. BLAST Analysis

The candidate primer/probe sets are submitted to BLAST analysis to check for possible overlap with any published sequences that might be missed by the Include/Exclude function. It also provides a useful summary.

### B. Secondary Structure

The methods of the present invention include analysis of nucleic acid secondary structure. This includes the structures of the primers and/or probes, as well as their intended target strain sequences. The methods and software of the invention predict the optimal temperatures for annealing, but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in the amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers).

### C. Evaluating the Primer and Probe Sequences

The methods and software of the invention may also analyze any nucleic acid sequence to determine its suitability in a nucleic acid amplification-based assay. For example, it can accept a competitor's primer set and determine the following information: (1) How it compares to the primers of the invention (e.g., overall rank, PCR and conservation ranking, etc.); (2) How it aligns to the exclude libraries (e.g., assessing cross-hybridization)—also used to compare primer and probe sets to newly published sequences; and (3) If the sequence has been previously published. This step requires keeping a database of sequences published in scientific journals, posters, and other presentations.

### Example 3

### Multiplexing

The Exclude/Include capability is ideally suited for designing multiplex reactions. The parameters for designing multiple primer and probe sets adhere to a more stringent set of parameters than those used for the initial Exclude/Include function. Each set of primers and probe, together with the resulting amplicon, is screened against the other sets that constitute the multiplex reaction. As new targets are accepted, their sequences are automatically added to the Exclude category.

The database is designed to interrogate the online databases to determine and acquire, if necessary, any new sequences relevant to the targets. These sequences are evaluated against the optimal primer/probe set. If they represent a new genotype or strain, then a multiple sequence alignment may be required.

### Example 4

### Sequences Identified for Detecting HSV-1 and/or HSV-2

The set of primers and probes were then scored according to the methods described herein to identify the optimized primers and probes of Table 3. It should be noted that the primers, as they are sequences that anneal to a plurality of identified or unidentified HSV-1 and/or HSV-2, can also be used as probes either in the presence or absence of amplification of a sample.

TABLE 3
Optimized Primers and Probes for the Detection of HSV-1 and/or HSV-2.

| | | | |
|---|---|---|---|
| Group | | | |
| No. | Forward Primer | Probe | Reverse Primer |

HSV-1

| | | | |
|---|---|---|---|
| 1 | ACCATCGCTTGGTTTCGG | AGGCAACTGTGCTATCCCCA | CCCCAGAGACTTGTTGTAGG |
| | SEQ ID NO: 1 | SEQ ID NO: 2 | SEQ ID NO: 3 |
| | ACCATCGCTTGGTTTCGG | AGGCAACGGTGCTATCCCCA | CCCCAGAGACTTGTTGTAGG |
| | SEQ ID NO: 1 | SEQ ID NO: 4 | SEQ ID NO: 3 |
| 2 | GGAGGCAACTGTGCTATC | CCCATCACGGTCATGGAGTACACCGA | CCCCAGAGACTTGTTGTAGG |
| | SEQ ID NO: 5 | SEQ ID NO: 6 | SEQ ID NO: 3 |
| 3 | CCGAAGACGTCCGGAAA | AACTGTGCTATCCCCATCACGGTCA | CCCAGAGACTTGTTGTAGGA |
| | SEQ ID NO: 7 | SEQ ID NO: 8 | SEQ ID NO: 9 |

HSV-2

| | | | |
|---|---|---|---|
| 4 | GAGATGCTGCTGGCCTTCA | TGACCTTCGTCAAGCAGTACGGCCC | CCTTGTAGATCTCCGTCAGCTT |
| | SEQ ID NO: 10 | SEQ ID NO: 11 | SEQ ID NO: 12 |
| 5 | CCACCTCCTCGATCGAGTT | CGCTGTATGTGGTTATACGTAAACTGCAGTCG | TGCGCCCCAGCATGTC |
| | SEQ ID NO: 13 | SEQ ID NO: 14 | SEQ ID NO: 15 |
| 6 | GTCCGCTCCGGAGAAGAC | TCCCTGTGTCGGCCACCGC | GCGCTTGGGTCGACTGAGG |
| | SEQ ID NO: 16 | SEQ ID NO: 17 | SEQ ID NO: 18 |
| 7 | GTCCGCTCCGGAGAAGAC | TCCCTGTGTCGGCCACCGC | GTCGGTTCCGCGCTTG |
| | SEQ ID NO: 16 | SEQ ID NO: 17 | SEQ ID NO: 19 |

A PCR primer set for amplifying an HSV-1 virus comprises at least one of the following sets of primer sequences: (1) SEQ ID NOS: 1 and 3; (2) SEQ ID NOS: 5 and 3; and (3) SEQ ID NOS: 7 and 9. A probe for binding to an amplicon(s) of an HSV-1 virus comprises at least one of the following probe sequences: SEQ ID NO: 2, 4, 6 and 8.

A PCR primer set for amplifying an HSV-2 virus comprises at least one of the following sets of primer sequences: (1) SEQ ID NOS: 10 and 12; (2) SEQ ID NOS: 13 and 15; (3) SEQ ID NOS: 16 and 18 and (4) SEQ ID NOS: 16 and 19. A probe for binding to an amplicon(s) of an HSV-2 virus comprises at least one of the following probe sequences: SEQ ID NOS: 11, 14 and 17.

The probes can be molecular beacon probes, TaqMan® probes, BHQ+ probes, and/or probes modified with locked nucleic acids.

The probes of the present invention are not limited to the modifications described herein. The probes of the present invention may be modified or unmodified.

Any set of primers can be used simultaneously in a multiplex reaction with one or more other primer sets, so that multiple amplicons are amplified simultaneously or can be used in a singleplex.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims. The contents of all references cited herein are incorporated by reference in their entireties.

What is claimed is:

1. An isolated nucleic acid sequence comprising a sequence selected from the group consisting of: SEQ ID NOS: 1-19.

2. A method of hybridizing one or more isolated nucleic acid sequences each comprising a sequence selected from the group consisting of: SEQ ID NOS: 1-19 to an HSV-1 and/or HSV-2 virus, comprising contacting the one or more isolated nucleic acid sequences to a sample comprising the HSV-1 and/or HSV-2 virus(es) under conditions suitable for hybridization.

3. The method of claim 2, wherein the HSV-1 and/or HSV-2 virus(es) is a genomic sequence, in a naturally occurring plasmid, in a naturally occurring transposable element, a template sequence or a sequence derived from an artificial construct.

4. The method of claim 2, further comprising isolating the hybridized HSV-1 and/or HSV-2 virus(es).

5. The method of claim 2, further comprising sequencing the hybridized HSV-1 and/or HSV-2 virus(es).

6. A primer set comprising at least one forward primer selected from the group consisting of SEQ ID NOS: 1, 5 and 7 (HSV-1) 10, 13 and 16 (HSV-2); and at least one reverse primer comprising a sequence selected from the group consisting of: SEQ ID NO: 3 and 9 (HSV-1); 12, 15, 18 and 19 (HSV-2).

7. A method of producing a nucleic acid product, comprising contacting one or more isolated nucleic acid sequences selected from the group consisting of SEQ ID NOS: 1-19 to a sample comprising an HSV-1 and/or HSV-2 virus(es) under conditions suitable for nucleic acid polymerization.

8. The method of claim 7, wherein the nucleic acid product is an amplicon produced using at least one forward primer selected from the group consisting of: SEQ ID NOS: 1, 5 and 7 (HSV-1) 10, 13 and 16 (HSV-2); and at least one reverse primer comprising a sequence selected from the group consisting of: SEQ ID NOS: 3 and 9 (HSV-1); 12, 15, 18 and 19 (HSV-2).

9. A probe that hybridizes to the nucleic acid product of claim 8.

10. The probe of claim 9, wherein the probe comprises a sequence selected from the group consisting of: SEQ ID NOS: 2, 4, 6 and 8 (HSV-1); 11, 14 and 17 (HSV-2).

11. The probe of claim 9, wherein the probe is labeled with a detectable label selected from the group consisting of: a fluorescent label, a chemiluminescent label, a quencher, a radioactive label, biotin and gold.

12. A set of probes that hybridize to the amplicon of claim 8, wherein a first probe comprises a sequence selected from the group consisting of: SEQ ID NOS: 2, 4, 6 and 8 (HSV-1) and a second probe comprises a sequence selected from the group consisting of: SEQ ID NOS: 11, 14 and 17 (HSV-2).

13. The set of probes of claim 12, wherein the first probe is labeled with a first detectable label and the second probe is labeled with a second detectable label.

14. The set of probes of claim 13, wherein the detectable labels are selected from the group consisting of: a fluorescent label, a chemiluminescent label, a quencher, a radioactive label, biotin and gold.

15. A method for detecting HSV-1 and/or HSV-2 in a sample, comprising:
a) contacting the sample with at least one forward primer comprising a sequence selected from the group consisting of: SEQ ID NOS: 1, 5 and 7 (HSV-1) 10, 13 and 16 (HSV-2); and at least one reverse primer comprising a sequence selected from the group consisting of: SEQ ID NOS: 3 and 9 (HSV-1); 12, 15, 18 and 19 (HSV-2), under conditions such that nucleic acid amplification occurs to yield an amplicon; and
b) contacting the amplicon with one or more probes comprising one or more sequences selected from the group consisting of: SEQ ID NOS: 2, 4, 6 and 8 (HSV-1); 11, 14 and 17 (HSV-2),
under conditions such that hybridization of the probe to the amplicon occurs;
wherein hybridization of the probe is indicative of HSV-1 and/or HSV-2 in the sample.

16. The method of claim 15, wherein each of the one or more probes is labeled with a different detectable label.

17. The method of claim 15, wherein the sample is selected from the group consisting of: saliva, fluids collected from the ear, eye, mouth, and respiratory airways, sputum, tears, oropharyngeal swabs, nasopharyngeal swabs, throat swabs, nasopharyngeal aspirates, bronchoalveolar lavage fluid, skin swabs, lip swabs, genital swabs, rectal swabs, cerebrospinal fluid, anogenital or oral lesion swabs, bone marrow, nasal aspirates, nasal wash, and fluids and cells obtained by the perfusion of tissues of both human and animal origin.

18. The method of claim 15, wherein the sample is from a human.

19. The method of claim 15, wherein the sample is non-human in origin.

20. The method of claim 15, wherein the sample is derived from an inanimate object or environmental surface.

21. The method of claim 15, wherein the at least one forward primer, the at least one reverse primer and the one or more probes are selected from the group consisting of: (1) SEQ ID NOS: 1, 2 and 3; (2) SEQ ID NOS: 1, 4 and 3; (3) SEQ ID NOS: 5, 6 and 3; (4) SEQ ID NOS: 7, 8 and 9; (5) SEQ ID NOS: 10, 11 and 12; (6) SEQ ID NOS: 13, 14 and 15; (7) SEQ ID NOS: 16, 17 and 18 and (8) SEQ ID NOS: 16, 17 and 19.

22. A kit for detecting HSV-1 and/or HSV-2 virus in a sample, comprising one or more probes comprising a sequence selected from the group consisting of: SEQ ID NOS: 2, 4, 6 and 8 (HSV-1); 11, 14 and 17 (HSV-2).

23. The kit of claim 22, further comprising:
a) at least one forward primer or primer pair comprising the sequence selected from the group consisting of: SEQ ID NOS: 1, 5 and 7 (HSV-1), 10, 13 and 16 (HSV-2); and
b) at least one reverse primer or primer pair comprising the sequence selected from the group consisting of: SEQ ID NO: 3, 9 (HSV-1); 12, 15, 18 and 19 (HSV-2).

24. The kit of claim 23, further comprising reagents for sequencing HSV-1 and/or HSV-2 virus in the sample.

25. The kit of claim 22, further comprising an internal control probe, internal control forward primer and internal control reverse primers.

26. The kit of claim 22, wherein the one or more probes are labeled with different detectable labels.

27. A method of diagnosing a condition, symptom or disease in a human associated with an HSV-1 and/or HSV-2 virus comprising:
a) contacting a sample with at least one forward and reverse primer set selected from the group consisting of: 1) SEQ ID NOS: 1 and 3; (2) SEQ ID NOS: 5 and 3; (3) SEQ ID NOS: 7 and 9; (4) SEQ ID NOS: 10 and 12; (5) SEQ ID NOS: 13 and 15; (6) SEQ ID NOS: 16 and 18 and (7) SEQ ID NOS: 16 and 19;
b) conducting an amplification reaction, thereby producing an amplicon; and
c) detecting the amplicon using one or more probes selected from the group consisting of: SEQ ID NOS: 2, 4, 6 and 8 (HSV-1); 11, 14 and 17 (HSV-2);
wherein the detection of an amplicon is indicative of the presence of an HSV-1 and/or HSV-2 virus in the sample.

28. The method of claim 27, wherein the sample is selected from the group consisting of: saliva, fluids collected from the ear, eye, mouth, and respiratory airways, sputum, tears, oropharyngeal swabs, nasopharyngeal swabs, throat swabs, nasopharyngeal aspirates, bronchoalveolar lavage fluid, skin swabs, lip swabs, genital swabs, rectal swabs, cerebrospinal fluid, anogenital or oral lesion swabs, bone marrow, nasal aspirates, nasal wash, and fluids and cells obtained by the perfusion of tissues of both human and animal origin.

29. The method of claim 27, wherein the condition, symptom or disease in a human associated with an HSV-1 and/or HSV-2 virus is selected from the group consisting of: fever, sore throat, sore mouth, gingivial lesions, lip lesions, ulcerative lesions, vesicular lesions, gingivostomatitis, edema, localized lymphadenopathy, anorexia, malaise, pharyngitis, dysuria, macules, papules, genital ulcers, encephalitis, lethargy, seizures, keratoconjunctivitis, meningitis, myelitis and radiculitis.

30. A kit for amplifying and sequencing an HSV-1 and/or HSV-2 virus in a sample, comprising:
a) at least one forward primer comprising the sequence selected from the group consisting of: SEQ ID NOS: 1, 5 and 7 (HSV-1), 10, 13 and 16 (HSV-2);
b) at least one reverse primer comprising the sequence selected from the group consisting of: SEQ ID NO: 3 and 9 (HSV-1); 12, 15, 18 and 19 (HSV-2); and
c) reagents for the sequencing of amplified DNA fragments.

31. A method of diagnosing a condition, symptom or disease in a human associated with an HSV-1 and/or HSV-2 virus, comprising contacting a denatured target from a sample with one or more probes comprising a sequence selected from the group consisting of: SEQ ID NOS: 2, 4, 6 and 8 (HSV-1); 11, 14 and 17 (HSV-2); under conditions for hybridization to occur; wherein hybridization of the one or more probes to a denatured target is indicative of the presence of an HSV-1 and/or HSV-2 virus in the sample.

32. The method of claim 31, wherein the sample is selected from the group consisting of: saliva, fluids collected from the ear, eye, mouth, and respiratory airways, sputum, tears, oropharyngeal swabs, nasopharyngeal swabs, throat swabs, nasopharyngeal aspirates, bronchoalveolar lavage fluid, skin swabs, lip swabs, genital swabs, rectal swabs, cerebrospinal fluid, anogenital or oral lesion swabs, bone marrow, nasal aspirates, nasal wash, and fluids and cells obtained by the perfusion of tissues of both human and animal origin

33. The method of claim 31, wherein the condition, symptom or disease in a human associated with HSV-1 and/or HSV-2 virus is selected from the group consisting of: fever, sore throat, sore mouth, gingivial lesions, lip lesions, ulcerative lesions, vesicular lesions, gingivostomatitis, edema, localized lymphadenopathy, anorexia, malaise, pharyngitis, dysuria, macules, papules, genital ulcers, encephalitis, lethargy, seizures, keratoconjunctivitis, meningitis, myelitis and radiculitis.

34. A probe that hybridizes to an HSV-1 and/or HSV-2 virus.

35. The probe of claim 34, wherein the probe comprises a sequence selected from the group consisting of: SEQ ID NOS: 2, 4, 6 and 8 (HSV-1); 11, 14 and 17 (HSV-2).

36. The probe of claim 34, wherein the probe is labeled with a detectable label selected from the group consisting of: a fluorescent label, a chemiluminescent label, a quencher, a radioactive label, biotin and gold.
